(19) United States
(12) Patent Application Publication
(10) Pub. No.: US 2007/0280926 A1
(43) Pub. Date: Dec. 6, 2007

(54) Sting kit apparatus and method of use

(76) Inventor: Lisa M. Ceko, Barrington Hills, IL (US)

Correspondence Address: Lesavich High-Tech Law Group, P.C., Suite 325, 39 S. LaSalle Street, Chicago, IL, 60603, US

(21) Appl. No.: 11/807,805

(22) Filed: May 30, 2007

Related U.S. Application Data
(60) Provisional application No. 60/810,104, filed on Jun. 1, 2006.

(51) Int. Cl. A61K 38/48; A61K 9/70; A61K 31/19; A61K 31/045

(52) U.S. Cl. 424/94.63; 514/557; 424/443; 514/724

(57) ABSTRACT

A sting kit apparatus and method of use. The apparatus is a portable kit that can be easily transported to a beach and can be used to immediately treat jelly fish stings or stings from other stinging organisms such as bees, wasps, hornets, etc. The kit includes a solution including a proteinase toxin neutralizer to neutralize jelly fish stinger toxin proteins and tools coated with proteolytic agents to attract, attach and further neutralize jelly fish stingers.

### CROSS REFERENCES TO RELATED APPLICATIONS

This application claims priority to U.S. Provisional Patent Application 60/810,104, filed Jun. 1, 2006, the contents of which are incorporated by reference.

### FIELD OF INVENTION

This application relates to sting kits. More specifically, it relates to a portable sting kit for jellyfish and other stinging organisms.

### BACKGROUND OF THE INVENTION

Jellyfish are free-swimming gelatinous creatures with hollow tentacles with stinging units that may be more than three feet in length. Jellyfish are typically found in shallow salt water and the stinging units (i.e., nematocysts) are capable of piercing the skin of human beings and transfer venom. More than one hundred species of jellyfish are toxic to humans. Waders often step on jellyfish with bare feet. Swimmers, surfers snorkelers and scuba divers come in contact with jellyfish in the water.

In the United States, Jellyfish stings occur most commonly during the summer along coastal regions. It is estimated that there about 500,000 jellyfish stings annually in the Chesapeake Bay area about 200,000 annually along the Florida coast.

Jellyfish stings also occur in internationally in tropical oceans, especially between about 30° south to 45° north in latitude. For example, there are a large number of jellyfish stings Australia during the warm summer months between November and May.

Contact with jellyfish venom causes a wide range of conditions, from skin rashes to cardiovascular and respiratory collapse. Almost immediately, painful raised red lesions (i.e., papules) develop in lines consistent with tentacle contact. The raised red lesions may develop into fluid filled lesions (vesicles) and may heal with pigmentation if left untreated. Muscle spasms may develop in the affected area. With extensive or repeated exposures systemic reactions can take place, such as; nausea, vomiting, headaches, dizziness (vertigo), muscle weakness and irregular heart rate (arrhythmias) may occur.

Immediate acute reactions to the venom tend to be toxic rather than allergic. Pain occurs immediately after exposure. The more rapidly the venom gets into the bloodstream, the higher the venom concentration in blood and the more rapid the onset of systemic symptoms.

There are several problems associated with jellyfish stings. One problem is that people who receive jellyfish stings are typically far away from any medical care. In some instances, a person with a jellyfish sting may be in the proximity of a life guard tower, which may, or may not have a first aid kit. The more immediate the medical care is the less severe the symptoms may be. Another problem is that a person with a jellyfish sting may have a severe reaction and go into shock and require immediate emergency care.

Thus, it would be desirable to solve some of the problems associated with jellyfish stings.

### SUMMARY OF THE INVENTION

In accordance with preferred embodiments of the invention, some of the problems associated with jellyfish stings are overcome.

A sting kit apparatus and method of use are presented. The apparatus is a portable kit that can be easily transported to a beach and can be used to immediately treat jelly fish stings or stings from other stinging organisms. The kit includes a solution including a proteinase toxin neutralizer to neutralize jelly fish stinger toxin proteins and tools coated with proteolytic agents to attract, collect and further neutralize jelly fish stingers.

The foregoing and other features and advantages of preferred embodiments of the present invention will be more readily apparent from the following detailed description. The detailed description proceeds with references to the accompanying drawings.

### BRIEF DESCRIPTION OF THE DRAWINGS

Preferred embodiments of the present invention are described with reference to the following drawings, wherein:

FIG. 1 is a block diagram illustrating a jelly fish sting kit; and

FIG. 2 is a flow diagram illustrating a method of using a jelly fish sting kit.

### DETAILED DESCRIPTION OF THE INVENTION

### Jelly Fish Sting Kit Apparatus

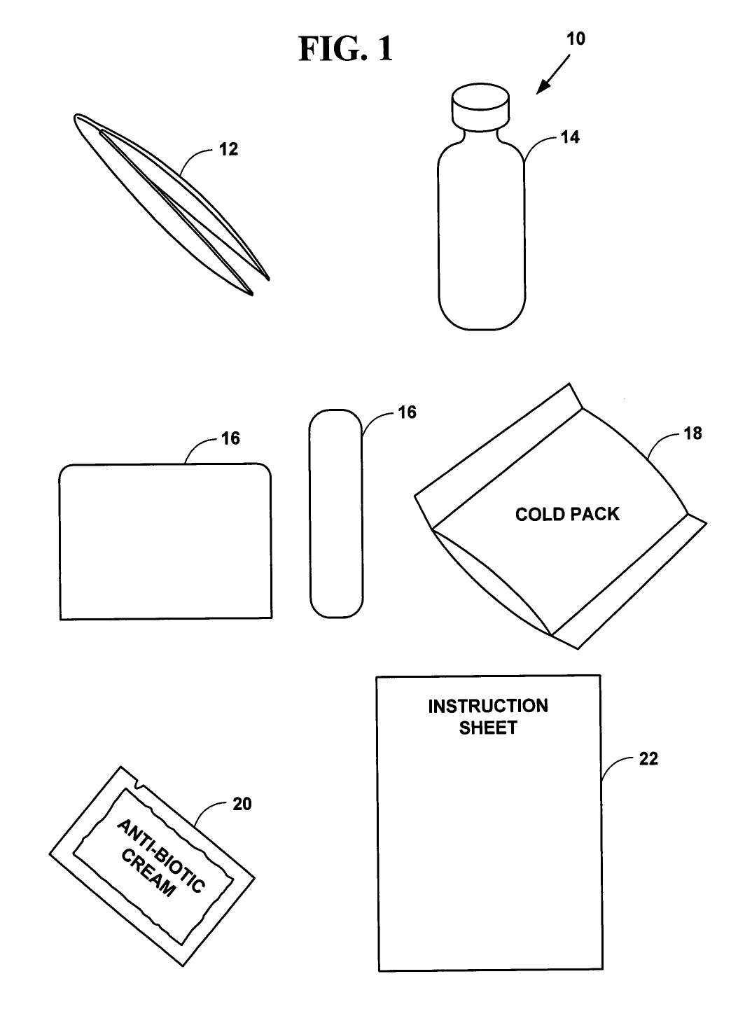

FIG. 1 is a block diagram illustrating a sting kit apparatus 10. The sting kit apparatus includes, but is not limited to, a vile 12 with a pre-determined neutralizing solution, a tweezers 14, a scraper 16, a cold pack 18, a package of anti-biotic cream 20 and an instruction sheet 22. However, the present invention is not limited to such an embodiment and more, fewer of other components can be used to practice the invention.

These components are included in a waterproof paper or plastic box or plastic bag to be placed into a backpack, purse, etc. for easy transport to the water. The apparatus 10 allows jelly fish strings to be immediately treated on or near the water. In one embodiment, the sting kit apparatus 10 is disposable and not reusable. In another embodiment, the sting kit apparatus 10 is reusable. In such an embodiment, the tweezers 14 and the scraper 16 are cleaned and sanitized between uses.

In one embodiment, the vile 12 includes an acetic acid solution. In one embodiment, the vile 12 includes about 5%-10% acetic acid solution used to pour over a jelly fish wound. Acetic acid inhibits further venom toxin discharge.

In another embodiment, the vile 12 includes a 50-% to 70% isopropyl alcohol solution or a papain solution. Alcohol and papain help neutralize protinase toxins in the venom.

The acetic acid changes the pH (i.e., measure of acidity or alkalinity of a solution) of the environment which denatures the protein toxin by changing its protein configuration to a less harmful state. The alcohol and papain changes the chemical structure of the venom toxin proteins. The alcohol and papain may also alter the pH of the environment.

As is a known in the art, “papain” is a cysteine protease with 212 amino acids stabilized by 3 disulfide bridges. Papains are present in a natural form in papayas.

Papains catalyze the breakdown of proteins by hydrolysis (i.e., addition of a water molecules). Papain is soluble in water at about 10 mg/ml. Immediately prior to use, the enzyme is typically diluted in a buffer containing about 5 mM L-cysteine.

Activation/stabilizing agents used with papain include EDTA, cysteine, and dimercaptopropanol or other activation/stabilizing agents.

The tweezers 14 are used to attract, collect, neutralize and remove any tentacles parts that remain on the wound. In one embodiment, tweezers 14 are metal, plastic, wood, rubber, composite or other material. However, the present invention is not limited to these materials and the tweezers 14 can comprise other materials.

In one embodiment the tweezers 14 are coated with a proteolytic agent compound to attract, collect and neutralize stingers from a jelly fish. The proteolytic compound attracts jelly fish stingers to the set of tweezers 14 to provide easier removal from a jelly fish sting wound on a human. The proteolytic compound also assists in neutralizing the jelly fish venom by breaking down the proteins associated with jelly fish stingers. In another embodiment, the tweezers 14 are not coated with a proteolyic agent.

The tweezers 14 allows the jellyfish stingers to be removed without release of the venom toxins from the stringers.

The scraper 16 is used to attract, collect and neutralize and scrap any remaining jelly fish parts such as tentacle barbs from the wound surface. In one embodiment, scraper 16 comprises metal, plastic, wood, rubber, composite or other material. However, the present invention is not limited to these materials and the scraper 16 can comprise other materials.

In one embodiment, the scraper 16 includes a shape of a credit card. In another embodiment, the scraper 16 includes a shape like a tongue depressor. However, the present invention is not limited to these shapes and other shapes can also be used to practice the invention. The scraper 16 allows the jellyfish stingers to be removed without release of the venom toxins from the stringers.

In one embodiment the scraper 16 is coated with a proteolytic agent compound to attract and further neutralize stingers from a jelly fish. In another embodiment, the scraper 16 is not coated with a proteolytic agent.

In one embodiment, the scraper 16 is coated with same proteolyitc agent as the tweezers 14. In another embodiment, the scraper 16 is coated with a different proteolytic agent than the tweezers 14.

As is known in the art, a “proteolyic agent” directs degradation of proteins by cellular enzymes called “proteases” or by intramolecular digestion. Proteases are enzymes which break peptide bonds of proteins. The proteolyic agent is specifically and chemically designed to attract, collect and degrade the proteins associated with jelly fish stingers and the toxins associated with venom discharge.

In one embodiment, the proteolyic agent is a residue created from a solution comprising a human contact lens protein remover. In one embodiment, the human contact lens protein remover includes, but is not limited to, a solution comprising Palixatonium chloride (e.g., about 0.006%, etc.) and Lauroyl quaterinised protein (e.g., about 0.0085%, etc.) wherein the aqueous portion is removed leaving a residue compound.

In another embodiment, the proteolyic agent includes a residue made from a solution of Subtilisin A tablets (e.g., 0.4 mg/tablet, etc.) or directly from a Subtilisin A powder. As is known in the arts, Subtilisin A is a non-glycosylated single polypeptide chain without disulfide bonds. Subtilisin A is a member of the Serine S8 Endoproteinase family. It has broad specificity with a preference for a large uncharged residue in the P1 position. It hydrolyzes native and denatured proteins, and is typically active under alkaline conditions. Subtilisin A supplied as a powder includes high purity lyophilized powders essentially free of buffer salts. Other types of Subtilisin can also be used to practice the inventions.

In one embodiment, the proteloyic agent is chemically bonded (e.g. based on electrical charges of the residue compound) to the tweezers 14 and/or scraper 16. In another embodiment, the proteloyic agent is applied to the tweezers and/or scraper 16.

However, the invention is not limited to such human contact lens protein removers or Subtilisin in these concentrations and other human contact lens protein removers with other concentrations and other proteolyic agents can also be used to practice the invention.

The cold pack 18 is used to apply to the wound after scraping. The anti-biotic cream 20 is used to prevent infection on the wound. The instruction sheet 22 includes instructions on proper use of the components of the sting kit apparatus 10.

In one embodiment, the anti-biotic cream 20 includes polymyxin B (e.g., 10,000 IU/g, etc.) bacitracin zinc (e.g., 500 IU/g, etc.), neomycin sulfate (e.g., 5 mg/g, etc.) or a combination thereof. However, the present invention is not limited to these anti-biotics and other anti-biotic creams can also be used to practice the invention.

In one embodiment, the anti-biotic cream 20 is replaced with a proteolyic agent cream. In another embodiment, the anti-biotic cream 20 is replaced with a hydrocortisone cream (e.g., 1%, etc.), benzocaine cream (e.g., 20%, etc.) or a combination thereof. Such creams help reduce or eliminate itching and swelling from the sting. However, the present invention is not limited to these anti-itching and anti-inflammatory creams and other types of anti-itching or anti-inflammatory creams or liquids can be used to practice the invention.

The sting kit apparatus 10 is described for jelly fish stings. However, the sting kit can also be used for other types of stings, such as insect stings (e.g., bees, wasps, hornets, etc.) and other types of stings from stinging organisms (e.g., scorpions, etc.). In such embodiments, the vile 12 will include appropriate neutralizing treating solutions, the tweezers 14 and/or the scraper 16 will be coated with and include appropriate proteolyic agents and the instruction sheet 22 will include other instructions specifically selected for the organism and type of sting components it may deposit in a wound on a human or animal such as a human pet or zoo animal.

### Jelly Fish Sting Kit Method of use

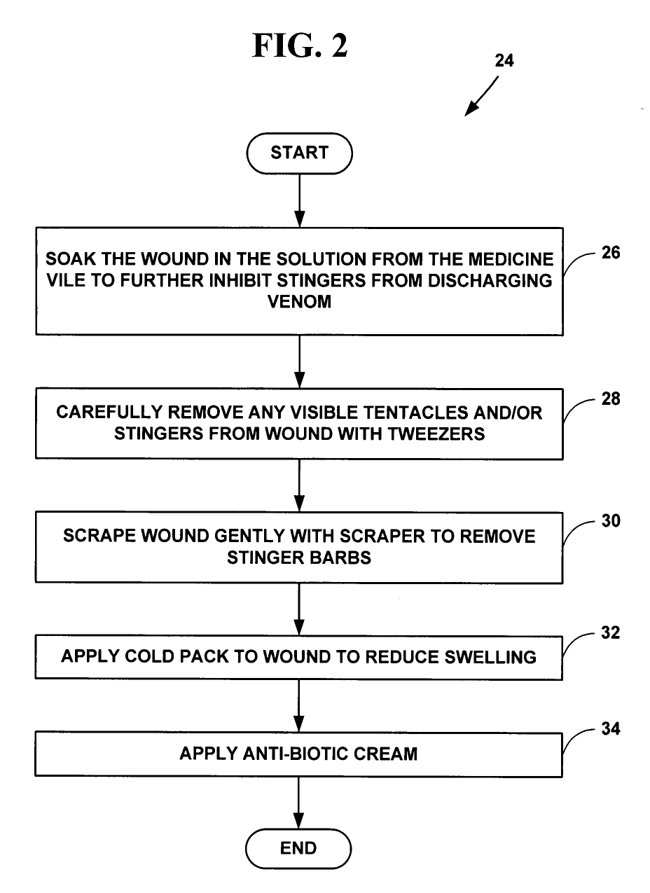

FIG. 2 is a flow diagram illustrating a Method 24 for using jelly fish sting kit. At Step 26, the vile 12 is opened and the wound is soaked for at least five minutes to further inhibit stingers from discharging venom. At Step 28, any visible tentacles and/or stingers are removed from the wound with the coated tweezers 14. At Step 30, the wound is gently scrapped with the coated scrapper 16 to remove any remaining stinger pieces, if any, from the wound. At Step 32, the cold pack 18 is applied for at least fifteen minutes to reduce swelling in and around the wound. At Step 34, the anti-biotic cream 20 is applied to the wound to prevent infection. However, the present invention is not limited to this method and other methods may be used to practice the invention.

In another embodiment, Steps 28 and 30 are practiced before Step 26. In such an embodiment, the tweezers 14 and scraper 16 are coated with the proteolyic agent are used to first neutralize the proteins associated with jellyfish stinger venom toxins.

However, the present invention is not limited to this method and other methods may be used to practice the invention.

The method and apparatus described herein are used for humans. However the method and apparatus can also be used for animals such as human pets, zoo animals or wild animals that may become stung by a stinging organism.

It should be understood that the processes, methods and system described herein are not related or limited to any particular type of component unless indicated otherwise. Various combinations of general purpose, specialized or equivalent components combinations thereof may be used with or perform operations in accordance with the teachings described herein.

In view of the wide variety of embodiments to which the principles of the present invention can be applied, it should be understood that the illustrated embodiments are exemplary only, and should not be taken as limiting the scope of the present invention. For example, the steps of the flow diagrams may be taken in sequences other than those described, and more or fewer or equivalent elements may be used in the block diagrams.

The claims should not be read as limited to the elements described unless stated to that effect. In addition, use of the term “means” in any claim is intended to invoke 35 U.S.C. §112, paragraph 6, and any claim without the word “means” is not so intended.

Therefore, all embodiments that come within the scope and spirit of the following claims and equivalents thereto are claimed as the invention.

I claim:

1. A jelly fish sting treating apparatus, comprising in combination:
a vile including a pre-determined neutralizing solution specifically chosen to neutralize proteins associated with jelly fish stingers and;
a tweezers coated with a pre-determined compound to attract and neutralize stingers from a jelly fish sting to allow easier removal from a jelly fish sting wound;
a scraper coated with a pre-determined compound to attract and neutralize stingers from a jelly fish sting to allow easier removal from a jelly fish sting wound;
a cold pack to reduce swelling from the jelly fish sting;
a package of anti-biotic cream; and
an instruction sheet.

2. The jellyfish sting treating apparatus of claim 1 wherein the pre-determined solution includes an acetic acid solution used to pour over a jelly fish wound, wherein acetic acid inhibits further venom discharge from jelly fish stingers.

3. The jellyfish sting treating apparatus of claim 2 wherein the acetic acid solution is about a 5%-10% acetic acid solution.

4. The jellyfish sting treating apparatus of claim 1 wherein the predetermined solution includes an isopropyl alcohol solution, wherein the alcohol neutralizes proteinase toxins in the venom released by jelly fish stingers.

5. The jellyfish sting treating apparatus of claim 4 wherein isopropyl alcohol solution is a 50%-70% isopropyl alcohol solution.

6. The jellyfish sting treating apparatus of Claiml wherein the pre-determined solution includes a papain solution, wherein the papain solution neutralizes proteinase toxins in the venom released by jelly fish stingers.

7. The jellyfish sting treating apparatus of claim 1 wherein the set of tweezers is coated with a pre-determined compound comprising a proteolytic agent.

8. The jelly fish sting treating apparatus of claim 1 wherein the scraper is coated with a pre-determined compound comprising a proteolytic agent.

9. The jelly fish sting treating apparatus of claim 1 wherein the pre-determined compound includes a proteolytic agent comprising a residue of a Subtilisin solution.

10. The jelly fish sting treating apparatus of claim 1 wherein the wherein the pre-determined compound includes a proteolytic agent comprising a residue of a protein removal solution for human contact lenses.

11. The jelly fish sting treating apparatus of claim 10 wherein the protein removal solution for human contact lenses includes Palixatonium chloride and Lauroyl quaterinised protein.

12. A jelly fish sting treating apparatus, comprising in combination:
means for neutralizing proteins associated with venom toxins from jelly fish stingers with a pre-determined neutralizing solution; and
means coated with a pre-determined compound for attracting, collecting and neutralizing proteins from jelly fish stingers to allow easier removal from a jelly fish sting wound.

13. The jelly fish sting treating apparatus of claim 12 wherein the means for neutralizing proteins includes a pre-determined neutralizing solution comprising acetic acid solution, isopropyl alcohol solution or a papain solution.

14. The jelly fish sting treating apparatus of claim 13 wherein the pre-determined neutralizing solution inhibits further venom discharge or neutralizes proteinase venom toxins released by jelly fish stingers.

15. The jelly fish sting treating apparatus of claim 12, wherein the means for attracting, collecting and neutralizing stingers includes a tweezers or a scraper coated with a proteolytic agent.

16. The jelly fish sting treating apparatus of claim 15 wherein the proteolytic agent is specifically and chemically designed to attract, collect and degrade the proteins associated with jelly fish stingers.

17. The jelly fish sting treating apparatus of claim 15 wherein the proteolytic agent includes residue of a Subtilisin solution or a residue of protein removal solution for human contact lenses.

18. A sting treating apparatus, comprising in combination:
means for neutralizing proteins associated with a stinger from a stinging organism with a pre-determined neutralizing solution; and
means coated with a proteolytic agent for attracting, collecting and neutralizing stingers from a stinging organism to allow easier removal from sting wound.

19. The sting treating apparatus of claim 18 wherein the stinging organism includes, bees, wasps, hornets and scorpions.

20. The sting treating apparatus of claim 18 wherein t pre-determined neutralizing solution includes a pre-determined neutralizing solution specifically selected to neutralize the proteins associated toxins associated with stinger discharge of the stinging organism.

21. The sting treating apparatus of claim 18 wherein the proteolytic agent includes a proteolytic agent specifically selected to neutralize the proteins associated toxins associated with stinger discharge of the stinging organism.
